(19) United States
(12) Patent Application Publication
(10) Pub. No.: US 2011/0213250 A1
(43) Pub. Date: Sep. 1, 2011

(54) 3-D ULTRASOUND IMAGING WITH VOLUME DATA PROCESSING

(75) Inventors: Michael Vion, Lynnwood, WA (US); Allen Snook, Snohomish, WA (US); Rohit Garg, Kirkland, WA (US)

(73) Assignee: KONINKLIJKE PHILIPS ELECTRONICS N.V., Eindhoven (NL)

(21) Appl. No.: 13/062,734

(22) PCT Filed: Sep. 8, 2009
(86) PCT No.: PCT/IB2009/053912

§ 371 Date: Apr. 29, 2011

Related U.S. Application Data
(60) Provisional application No. 61/097,274, filed on Sep. 16, 2008.

(51) Int. Cl. A61B 8/14

(52) U.S. Cl. 600/443

(57) ABSTRACT

In an ultrasound imaging system, an ultrasound scanning assembly (USC) provides volume data (VD) resulting from a three-dimensional scan of a body (BDY). A region of interest detector (RDT) detects a region within the volume data (VD) characterized by a variation of at least one data parameter, which exceeds a margin. A slice generator (SLG) may then generates-slices (SX) from the region that has been detected. These slices (SX) can be displayed on a display device (DPL).

### FIELD OF THE INVENTION

An aspect of the invention relates to an ultrasound imaging system that is capable of carrying out a three-dimensional (3-D) ultrasound scan and processing volume data resulting from such a scan. The ultrasound imaging system may be helpful in, for example, fetal examinations or gallbladder examinations. Other aspects of the invention relate to a method of ultrasound imaging, and a computer program product.

### BACKGROUND OF THE INVENTION

A 3-D ultrasound scan typically involves emitting ultrasound waves that illuminate, as it were, a particular volume within a body, which may be designated as target volume. This can be achieved, for example, by emitting ultrasound waves at multiple different angles. Volume data is obtained by receiving and processing reflected waves. The volume data is a representation of the target volume within the body. The volume data can be displayed on a display device in a fashion that provides a three-dimensional representation, which gives an impression of width, height, and depth. In obstetric applications, it is possible to obtain a photo- or film-like image of a fetus with surface details that delineate facial, limbs and body features. This allows prospective parents to see and appreciate what physicians see.

Volume data can be of great diagnostic value because arbitrary slices can be taken from the volume data and visualized on the display device. Slicing can thus provide different views of the target volume, which allows a physician to study subtle anatomical structures in detail. The volume data may be stored so that the physician may manipulate this data to obtain any desired slice after a 3-D ultrasound scan of a patient and the patient is discharged. The physician may explore the target volume by, for example, scrolling through parallel planes and by rotating the target volume to obtain a view of an object of interest. Precise slicing allows the physician to display images that are difficult to achieve manually, or cannot be achieved at all. No human being could hold his or her hand still enough to sweep or acquire individual images in sufficiently fine slicing intervals or scan from a perspective of a third plane.

The article entitled “iSlice Ultrasound Image Display” in Medica Mundi, vol. 50, no. 3, 2006, pages 52 and 53, describes an ultrasound system designated “iU22” manufactured by Royal Philips Electronics. The article can be found under the URL: http://www.medical.philips.com/main/news/assets/docs/medicamundi/mm_vol50_no3/14_Te chnology_News.pdf).

The article mentions that finding the best views and content when capturing an ultrasound image can often be challenging for a sonographer. The iU22 ultrasound system provides volumetric imaging and slicing capabilities, which make it faster and easier to capture and find the best views for making a diagnosis. After acquiring a volume image with the iU22 ultrasound system, QLAB software can do precision slicing of the volume and display 4, 9, 16 or 25 20 images from the volume set. This slicing is referred to as “iSlice”. Clinicians can then examine the images from multiple angles and select the best images for further evaluation and reports. When rotating the volume the two-dimensional (2-D) views are instantaneously updated to reflect the new perspective. In addition, volumetric imaging with iSlice gives clinicians the ability to obtain additional views, e.g. coronal, which are unavailable with conventional 2-D imaging. This is very valuable when assessing complex pathologies. The sonographer is also able to adjust the amount of slices desired as well as the interval slicing in order to conform to different applications.

### SUMMARY OF THE INVENTION

There is a need for an improved ultrasound imaging system, which allows a precise and comprehensive analysis of volume data.

In order to better address this need, the following points have been taken into consideration. In conventional systems, a physician needs to cine through the volume data to find an object of interest, which may be analyzed through slicing. This operation may be relatively difficult to perform, even if the physician is trained and experienced, in particular when the target volume comprises relatively complex anatomical objects. In case there are several objects of interest, the physician may miss one of those.

In accordance with an aspect of the invention, an ultrasound imaging system comprises an ultrasound scanning assembly that provides volume data resulting from a three-dimensional scan of a body. The ultrasound imaging system further comprises a region of interest detector that detects a region within the volume data characterized by a variation of at least one data parameter, which exceeds a margin.

A variation of a data parameter at a particular location within the volume data may mark an object of interest, or a boundary thereof. Accordingly, it is possible to detect objects of interest within the volume data by detecting such variations. This can be done automatically by means of, for example, a processor into which suitable detection software has been loaded. Such an automatic detection may assist a physician in identifying objects of interest within the volume data. There will be less risk that the physician misses an object of interest. Moreover, detection of regions of interest in accordance with the invention alleviates the physician's task of manipulating and analyzing volume data.

An implementation of the invention advantageously comprises one or more of the following additional features, which are described in separate paragraphs that correspond with individual dependent claims.

The ultrasound system preferably comprises a slice generator that generates slices from the region that has been detected. These slices can be displayed on a display device.

The ultrasound system preferably comprises an interface via which an operator can specify the at least one data parameter used for detecting a region of interest.

The at least one data parameter used for detecting a region of interest preferably comprises a data parameter selected from the following group: average voxel magnitude, contrast, entropy, homogeneity.

The at least one data parameter may preferably comprise a set of parameters in the form of a histogram.

The region of interest detector may detect the region of interest by comparison of a set of global values, which have been determined for the at least one parameter based on the volume data in its entirety, with a set of local values, which have been determined for the at least one parameter based on a portion of the volume data.

The region of interest detector may detect the region of interest by comparison of a set of local values, which have been determined for the at least one parameter based on a portion of the volume data, with another set of local values, which have been determined for the at least one parameter based on another, adjacent portion of the volume data.

A detailed description, with reference to drawings, illustrates the invention summarized hereinbefore as well as the additional features.

### BRIEF DESCRIPTION OF THE DRAWINGS

FIG. 1 is a block diagram that illustrates an ultrasound imaging system.

FIG. 2 is a flow chart diagram that illustrates a series of steps that the ultrasound imaging system can carry out.

FIG. 3 is a flow chart diagram that illustrates an alternative series of steps that the ultrasound imaging system can carry out.

### DETAILED DESCRIPTION OF THE INVENTION

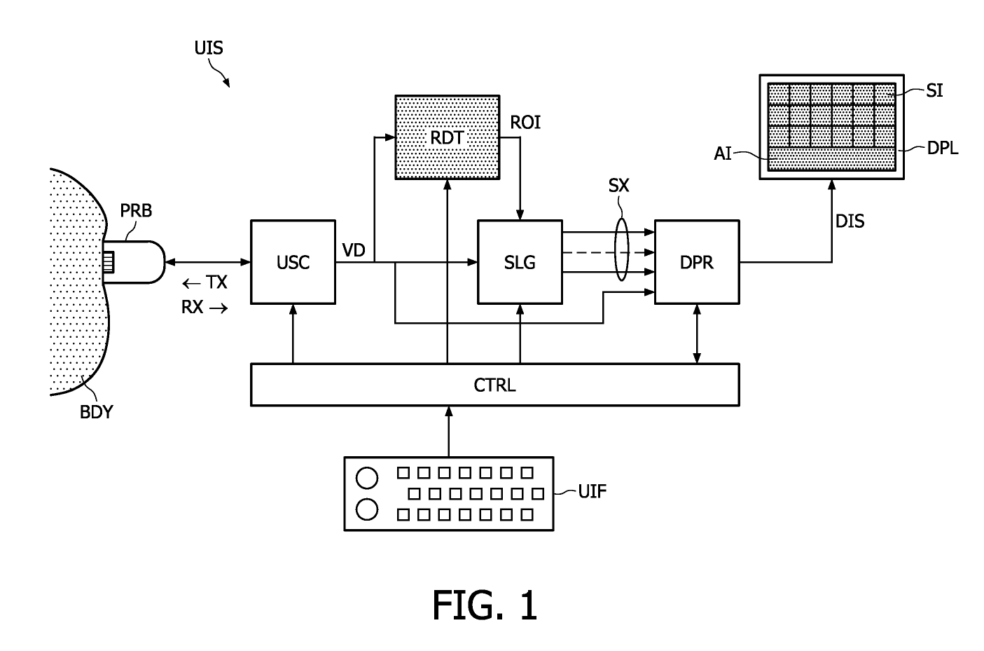

FIG. 1 illustrates an ultrasound imaging system UIS, which capable of carrying out a 3-D ultrasound scan. The ultrasound imaging system UIS comprises various functional entities that constitute an ultrasound imaging acquisition-and-processing path: a probe PRB, an ultrasound scanning assembly USC, a region of interest detector RDT, a slice generator SLG, and a display processor DPR. The probe PRB may comprise, for example, a two-dimensional array of piezoelectric transducers. The ultrasound scanning assembly USC may comprise an ultrasound transmitter and an ultrasound receiver, which may each include a beam-forming module. The ultrasound scanning assembly USC may further comprise one or more filter modules, a so-called B-mode processing module, and a Doppler-mode processing module.

The region of interest detector RDT may be implemented by means of, for example, a set of instructions that has been loaded into a programmable processor. In such a software-based implementation, the set of instructions defines operations that the region of interest detector RDT carries out, which will be described hereinafter. The same holds for other functional entities, such as, for example, the slice generator SLG, the display processor DPR, as well as one or more modules that functionally belong to the ultrasound scanning assembly USC. Each of these may also be implemented by means of a set of instructions, a software module, which has been loaded into a programmable processor.

The ultrasound imaging system further comprises a display device DPL, a controller CTRL, and a user interface UIF. The controller CTRL may be in the form of, for example, a suitably programmed processor. The user interface UIF may comprise physical elements, such as, for example, various alphanumerical keys, knobs, and a mouse or trackball. However, the user interface UIF may also comprise software components, which the controller CTRL carries out. For example, a software component may cause the display device DPL to display a menu from which an operator may select an item by pressing a particular key or by moving a cursor to the item as displayed.

The ultrasound imaging system UIS basically operates as follows. It is assumed that the probe PRB is in contact with a body BDY as illustrated in FIG. 1, which may be the body BDY of a patient. The ultrasound scanning assembly USC applies a set of transmission signals TX to the probe PRB. This causes the probe PRB to emit ultrasound waves into the body BDY that illuminate, as it were, a target volume. To that end, the probe PRB may emit, for example, ultrasound waves at multiple different angles. Alternatively, the set of transmission signals TX may cause the probe PRB to emit a relatively broad beam, which may be designated as a “fat” beam.

The probe PRB receives reflections of the ultrasound waves, which occur in the target volume within the body BDY. In response to these received reflections, the probe PRB provides a set of reception signals RX. The ultrasound scanning assembly USC processes the set of reception signals RX so as to obtain volume data VD. The volume data VD may be in the form of, for example, so-called B-mode 3-D image, or a 3-D Doppler-based image, which may comprise color information representing speed of movement. The volume data VD is typically composed of so-called voxels, which are elementary units similar to pixels, which constitute elementary units of a 2-D image.

The region of interest detector RDT processes the volume data VD so as to identify one or more regions of interest within the volume data VD. This identification is based on one or more data parameters, which may be predefined or which an operator may select by means of the user interface UIF. In broad terms, the region of interest detector RDT detects variations of the one or more data parameters concerned within the volume data VD. A region of interest is characterized by a variation of the one or more data parameters concerned, which exceeds a margin. The margin may be predefined or operator defined. The identification of regions of interest will be described in greater detail hereinafter. The region of interest detector RDT provides a region of interest indication ROI, which indicates respective locations of respective regions of interest that have been detected as such.

The slice generator SLG is capable of generating slices SX from the volume data VD. The slice generator SLG may do so in a fashion be similar to, for example, the iSlice feature in the iU22 ultrasound system mentioned hereinbefore. Importantly, the region of interest indication ROI guides, as it were, the slice generator SLG or the operator, or both, in generating slices SX from the volume data VD. Accordingly, the slices SX may be concentrated, as it were, and appropriately located in one or more regions of interest. This can be done in an automatic fashion or in a semi-automatic fashion.

The slice generator SLG may automatically locate slices SX within a region of interest that has been detected as such by the region of interest detector RDT. For example, in case the iSlice feature is applied, the slice generator SLG may automatically determine the location and the orientation of a reference plane on the basis of the region of interest indication ROI. The slices SX constitute planes that are parallel to the reference plane and that are equidistantly spaced. The slice generator SLG may automatically determine an appropriate equidistant spacing on the basis of the region of interest indication ROI. The slices SX that are thus automatically obtained may constitute an initial multi-slice view of the region of interest. The operator may then adjust, or rather fine-tune, the location and the orientation of the reference plane, as well as the equidistant spacing between the slices SX. The operator may thus obtain various different multi-slice views of the region of interest.

Alternatively, the operator may locate slices SX within a region of interest in a substantially manual fashion. To that end, the display processor DPR may provide a visual representation of the volume data VD, wherein regions of interest are marked. The region of interest indication ROI, which the region of interest detector RDT provides, allows such marking. The operator may then locate and orientate one or more planes, which represent slices SX to be taken, within the visual representation of the volume data VD. To that end, the controller CTRL may comprise an interactive slice definition software module that generates such planes and that allows the operator to manipulate these planes. Once the operator considers that the planes are appropriately located and oriented, he or she may indicate the same by, for example, depressing an “OK” button on the user interface UIF. In response, the controller CTRL applies to the slice generator SLG a definition of the location and the orientation of slices SX to be generated from the volume data VD.

The display processor DPR generates display images DIS that typically comprise a visual representation of the slices SX that the slice generator SLG has generated from the volume data VD. Each slice may be visualized by means of an individual sub-image SI in a display image DIS. Respective sub-images representing respective slices SX may be displayed side-by-side in the form of a matrix, as illustrated in FIG. 1, or any other form that the operator desires. The display image DIS may further comprise additional information AI relating to, for example, the location, the orientation, and the spacing of the slices SX. The display image DIS may further comprise a visual representation of the volume data VD, which may constitute an additional sub-image. As indicated hereinbefore, this visual representation may comprise additional elements that indicate actual or desired locations and orientations of slices SX within the volume data VD that are currently visualized, or that needs to be visualized, respectively.

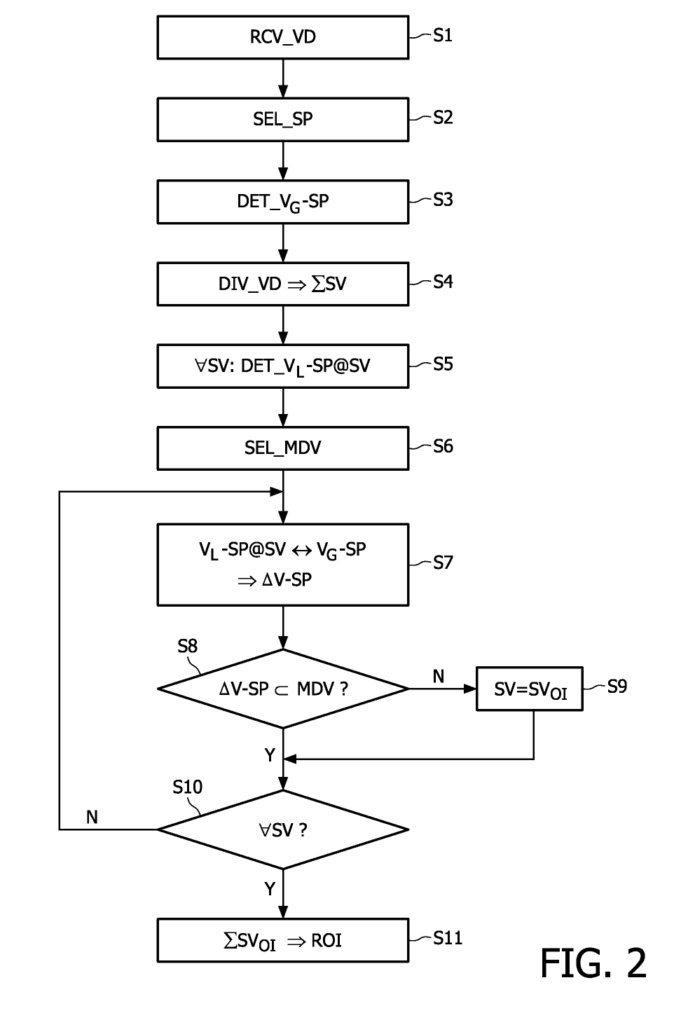

FIG. 2 illustrates a series of steps S1-S11 that the region of interest detector RDT may carry out in order to provide a region of interest indication ROI. As mentioned hereinbefore, the region of interest detector RDT may be implemented by means of a programmable processor. FIG. 2 may therefore be regarded as a flowchart representation of a software program, that is, a set of instructions, which enables the programmable processor to carry out various operations described hereinafter with reference to FIG. 2.

In step S1 (RCV_VD), the region of interest detector RDT receives the volume data VD that the ultrasound scanning assembly USC provides following a 3-D ultrasound scan. As mentioned hereinbefore, the volume data VD may be in the form of a 3-D image composed of voxels, which are elementary units similar to pixels, which constitute elementary units of a 2-D image. The volume data VD may comprise B-mode information, or Doppler information relating to speed of movement, or a combination of these types of information, as well as other information obtained by means of the 3-D ultrasound scan.

In step S2 (SEL_SP), the region of interest detector RDT obtains a set of data parameters, which are to be applied in detecting a region of interest. These data parameters will be referred to hereinafter the abbreviated term “parameters”. A parameter may relate to voxel magnitude, which corresponds with luminance and represents echo strength. A parameter may also relate to color when, for example, the volume data VD comprises Doppler information typically represented as color. A parameter typically relates to a plurality of voxels. Examples of such parameters include contrast, entropy, homogeneity, the last two mentioned parameters being of statistical nature. The set of parameters may be expressed in the form of a histogram or a set of histograms. The set of parameters comprise only a single parameter. That is, only one parameter may serve as a basis for detecting a region of interest. This parameter may be, for example, an average value of voxel magnitudes in a given volume.

The controller CTRL and the user interface UIF associated therewith may be arranged so that the operator may define the set of parameters that the region of interest detector RDT receives and will apply. For example, the controller CTRL may cause the display device DPL to display a menu from which the operator may select one or more parameters. The controller CTRL then applies selected parameters to the region of interest detector RDT. The set of parameters may also be predefined and, to that end, pre-programmed in a memory.

In step S3 (DET_V_{G}-SP), the region of interest detector RDT determines a set of global values for the set of parameters concerned. The set of global values are determined from the volume data VD in its entirety. For example, let it be assumed that average voxel magnitude is a parameter in the set of parameters. In that case, the region of interest detector RDT determines the average magnitude value for all voxels in the volume data VD, which value may be designated as the global average value. In case average voxel magnitude is the single parameter in the set of parameters, the set of global values will comprise only a single global value: the global average value. As another example, let it be assumed that the set of parameters is in the form of a histogram comprising several voxel magnitude ranges. In that case, region of interest detector RDT determines a number of voxels for each magnitude range taking into account all voxels in the volume data VD. The set of global values comprises the respective numbers that have been determined for the respective magnitude ranges within the histogram.

In step S4 (DIV_VD [symbol] ΣSV), the region of interest detector RDT effectively divides the volume data VD into a plurality of sub-volumes. A sub-volume may have the shape of, for example, a cube, or a pyramid, or any other suitable shape. Dividing the volume data VD into cubes may be compared with dividing a two-dimensional image into blocks. In a sense, the sub-volumes may be regarded as building blocks that collectively form the volume data VD. Each sub-volume constitutes a selection of voxels within the volume data VD that have neighboring locations.

In step S5 (∀SV: DET_V_{L}-SP@SV), the region of interest detector RDT determines a plurality of sets of local values for the set of parameters concerned. A set of local values is determined for a particular sub-volume on the basis of the voxels comprised within the sub-volume concerned. For example, in case average voxel magnitude is a parameter in the set of parameters, the region of interest detector RDT determines the average magnitude value for the voxels that are present within the sub-volume concerned. As another example, in case the set of parameters is in the form of a histogram, the region of interest detector RDT determines respective numbers of voxels for respective magnitude ranges in histogram. These respective numbers will then form part of the set of local values for the sub-volume concerned

In step S6 (SEL_MDV), the region of interest detector RDT obtains a deviation margin definition, which defines a margin of deviation from the set of global values. In a certain sense, the deviation margin definition defines a peripheral zone around the set of global values. For example, in case the set of global values comprises only a single global value, such as, for example, the global average value, the deviation margin definition may comprise a negative deviation margin and a positive deviation margin. The negative deviation margin and the positive deviation margin define a range of values that comprises the global average value. More specifically, the range of values has a lower boundary, which is equal to the global average value minus the negative deviation margin, and an upper boundary, which is equal to the global average value plus the positive deviation margin.

The deviation margin definition may further define a manner in which a set of local values should be compared with the set of global values. To that end, the deviation margin definition may comprise, for example, scaling and weighting coefficients. For example, let it be assumed that the set of global values comprises a global histogram based on all voxels in the volume data VD. Comparing this global histogram, in terms of shape, with a corresponding local histogram based on voxels in a sub-volume, may involve a scaling operation. For example, respective numbers in the global histogram may each be divided by a number corresponding to the number of sub-volumes comprised in the volume data VD. Weighting coefficient in the deviation margin definition may define a degree of weight that should be given to a number deviation in a particular voxel magnitude range

The deviation margin definition may be determined on the basis of the set of global values determined in step S3 and the plurality of sets of local values determined in step S5. This may involve a statistical analysis. For example, it may be desired that 10 to 20% of the sub-volumes are marked as a sub-volume of interest, as will be described hereinafter. Not too many or too few sub-volumes should be marked. This can be achieved by appropriately establishing the deviation margin definition, which may be done in an automatic or a semi-automatic fashion. For example, the region interest detector may autonomously determine the deviation margin definition based on the set of global values and the plurality of sets of local values.

As another example, the region of interest detector RDT, or any other functional entity, may cause the display device DPL to display one or more graphs representing the set of global values and sets of local values. These graphs may comprise visual elements representing a particular deviation margin definition that the operator has specified, as well as further visual indications illustrating statistical detection properties obtained by applying the deviation margin definition. The operator may then modify the deviation margin definition and observe effects thereof, so as to arrive at a suitable deviation margin definition.

In step S7 (V_{L}-SP@SV [symbol] V_{G}-SP [symbol] ΔV-SP), the region of interest detector RDT determines, for a particular sub-volume, a deviation of the set of local values, which have been determined for this sub-volume, with respect to the set of global values. This operation may be relatively straightforward in case the set of parameters concerned comprises the average voxel magnitude as the single parameter. In that case, the region of interest detector RDT may subtract a local average value from the global average value. A single difference value represents the deviation. In case a parameter is expressed in the form of a histogram, the deviation will typically comprise respective values for respective magnitude ranges. A value may represent a greater number or a smaller number of voxels in the magnitude range of interest with respect to a typical number expressed by the set of global values.

In step S8 (ΔV-SP⊂MDV?), the region of interest detector RDT determines whether the deviation is within the margin of deviation as defined by the deviation margin definition, or not. In case the deviation is within the margin (Y), the sub-volume concerned can be considered as “quite normal” or, differently stated, “not special” having regard to the volume data VD in its entirety, at least as far as the set of parameters are concerned. In that case, the region of interest detector RDT subsequently carries out step S10, which will be described hereinafter. In contrast, in case the deviation is outside the margin as defined by the deviation margin definition (N), the region of interest detector RDT subsequently carries out step S9.

In step S9 (SV=SV_{OI}), the region of interest detector RDT marks the sub-volume concerned as belonging to the category “of interest”. In a manner of speaking, the sub-volume concerned is considered as “special” in the sense that the set of local values, which have been determined for this sub-volume, differ to relatively great extent from the set of global values. The sub-volume is remarkable having regard to the set of parameters concerned.

In step S10 (∀SV?), the region of interest detector RDT checks whether steps S7 and S8 have been carried out for all sub-volumes, or not. In case there are one or more sub-volumes for which these steps have not yet been carried out, the region of interest detector RDT returns to step S7 and subsequently carries out the aforementioned steps for one such a sub-volume. In case steps S7 and S8 have been carried out for all sub-volumes, the region of interest detector RDT subsequently carries out step S11.

In step S11 (ΣSV_{OI} [symbol] ROI), the region of interest detector RDT detects one or more clusters of sub-volumes that have been marked as “of interest”. Such a cluster constitutes a region of interest within the volume data VD from which slices SX should preferably be generated. Stated differently, the region of interest detector RDT identifies a region of interest as a cluster of sub-volumes that each have local values that deviate to relatively great extent from the global values. The region of interest indication ROI indicates such regions of interest. As described hereinbefore, the slice generator SLG illustrated in FIG. 1 may use this information to generate the slices SX that are applied to the display processor DPR.

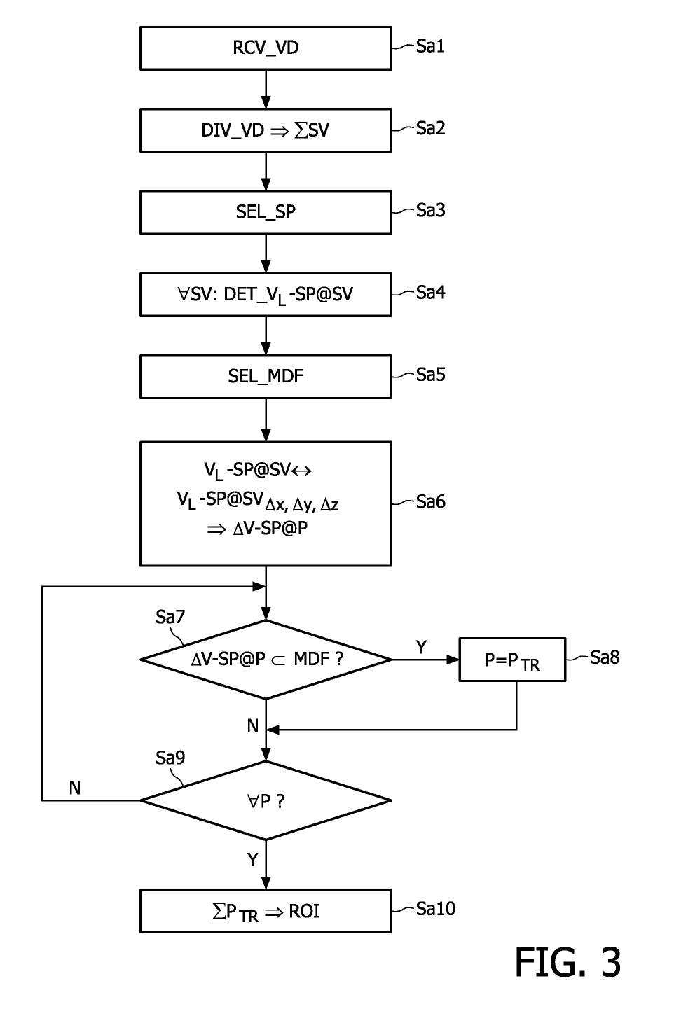

FIG. 3 illustrates an alternative series of steps that the region of interest detector RDT may carry out in order to provide a region of interest indication ROI. Similar to FIG. 2, FIG. 3 may also be regarded as a flowchart representation of a software program, that is, a set of instructions, which enables a programmable processor to carry out various operations described hereinafter with reference to FIG. 3.

In step Sa1 (RCV_VD), the region of interest detector RDT receives the volume data VD that the ultrasound scanning assembly USC provides following a 3-D ultrasound scan. Similar remarks apply as those made hereinbefore with regard to step S1 illustrated in FIG. 2.

In step Sa2 (DIV_VD [symbol] ΣSV), the region of interest detector RDT effectively divides the volume data VD into a plurality of sub-volumes. Similar remarks apply as those made hereinbefore with regard to step S4 illustrated in FIG. 2.

In step Sa3 (SEL_SP), the region of interest detector RDT obtains a set of parameters, which are to be applied in detecting a region of interest. Similar remarks apply as those made hereinbefore with regard to step S2 illustrated in FIG. 2.

In step Sa4 (∀SV: DET_V_{L}-SP@SV), the region of interest detector RDT determines a plurality of sets of local values for the set of parameters concerned. A set of local values is determined for a particular sub-volume on the basis of the voxels comprised within the sub-volume concerned. Similar remarks apply as those made hereinbefore with regard to step S5 illustrated in FIG. 2.

In step Sa5 (SEL_MDF), the region of interest detector RDT obtains a difference margin definition, which defines a margin of difference between two respective sets of local values of two respective neighboring sub-volumes. For example, in case the set of parameters comprises only a single parameter, such as, for example, average voxel magnitude, the difference margin definition may comprise single step-size value. The difference margin definition may be defined in various different manners similar to those described hereinbefore with regard to the deviation margin definition, which is determined in step S6 illustrated in FIG. 2.

The difference margin definition may further define a manner in which the difference should be established between two respective sets of local values of two respective made in step. To that end, the difference margin definition may comprise, for example, weighting coefficients. For example, let it be assumed that each set of local values comprises a histogram. Weighting coefficient in the deviation margin definition may define a degree of weight that should be given to a number difference in a particular voxel magnitude range

In step Sa6 (V_{L}-SP@SV [symbol] V_{L}-SP@SV_{Δx, Δy, Δz} [symbol] ΔV-SP@P), the region of interest detector RDT determines, for a sub-volume, respective sets of difference values with regard to respective neighboring sub-volumes. The region of interest detector RDT may do so for each sub-volume in the volume data VD. A set of difference values can be associated with a boundary plane of a sub-volume, as well as a corresponding boundary plane of a neighboring sub-volume that touch each other, as it were.

For example, let it be assumed that sub-volumes are in the shape of a cube. In that case, a sub-volume has six boundary planes that may be designated as follows: a left plane, a right plane, an upper plane, a lower plane, a front plane, and a back plane. The left plane of a sub-volume may touch the right plane of another, neighboring sub-volume. A set of difference values can be determined for these planes, which corresponds to differences between the respective sets of local values of the two sub-volumes concerned. For example, let it be assumed that the set of parameters comprises only a single parameter, such as, for example, average voxel magnitude. In that case, the set of difference values may comprise a single value only, which represents a difference between the respective local average magnitude values of the two sub-volumes concerned.

In step Sa7 (ΔV-SP@P⊂MDF?), the region of interest detector RDT determines for a boundary plane whether the set of difference values associated with the boundary plane is within the margin of difference as defined by the defense margin definition, or not. In case the sets of difference values for a boundary plane is within the margin, the set of parameters can be considered undergoing a modest variation between the two sub-volumes neighboring of interest. There is no sharp transition. In that case, the region of interest detector RDT subsequently carries out step Sa9, which will be described hereinafter. In contrast, in case the set of difference values is outside the margin as defined by the difference margin definition, the region of interest detector RDT subsequently carries out step Sa8.

In step Sa8 (P=P_{TR}), the region of interest detector RDT marks the boundary plane concerned as belonging to the category “transition”. Since the set of difference values that have been determined for this boundary plane exceeds the margin of difference, the boundary plane concerned is considered as constituting a transition within the volume data VD.

In step Sa9 (∀P?), the region of interest detector RDT checks whether step Sa1 has been carried out for all boundary planes, or not. In case there are one or more boundary planes for which step Sa7 has not yet been carried out, the region of interest detector RDT returns to this step. Step Sa7 is subsequently carried out and, if applicable, step Sa8 for one such a sub-volume. In case step S7 has been carried out for all boundary planes, the region of interest detector RDT subsequently carries out step Sa10.

In step Sa10 (ΣP_{TR} [symbol] ROI), the region of interest detector RDT detects one or more groups of transition boundary planes that substantially form an outline, or rather surface, delimiting a region within the volume data VD. Such a region constitutes a region of interest within the volume data VD from which slices SX should preferably be generated. Stated differently, the region of interest detector RDT identifies a region of interest as being substantially delimited by a group of boundary planes that constitute a circumference of the region of interest concerned. The region of interest indication ROI indicates such regions of interest. As mentioned hereinbefore, the slice generator SLG illustrated in FIG. 1 may use this information to generate the slices SX that are applied to the display processor DPR.

### CONCLUDING REMARKS

The detailed description hereinbefore with reference to the drawings is merely an illustration of the invention and the additional features, which are defined in the claims. The invention can be implemented in numerous different ways. In order to illustrate this, some alternatives are briefly indicated.

The invention may be applied to advantage in numerous types of products or methods related to volumetric ultrasound imaging. For example, the invention may be applied in a portable computer, which is configured for volumetric ultrasound imaging purposes. The portable computer may interface with, for example, a dedicated ultrasound imaging module that comprises, for example, one or more beamformers as well as other circuits for applying activation signals to a probe and for processing reception signals from the probe. Such a dedicated ultrasound imaging module will typically comprise analog to digital converters and digital to analog converters.

There are numerous ways in which an ultrasound imaging system in accordance with the invention may detect a region within the volume data characterized by a variation of at least one data parameter, which exceeds a margin. For example, the volume data may initially be divided into relatively large sub-volumes so as to detect which of these relatively large sub-volumes are of interest. Subsequently, these relatively large volumes of interest may be divided into smaller sub-volumes so as to detect which of these smaller sub-volumes are of interest. That is, detection of a region of interest may involve a hierarchy of detection levels, which are gradually gone through, starting at a coarse detection level and ending at a fine detection level. Such a hierarch-based detection may be more efficient than the methods illustrated in FIGS. 2 and 3, which may be regarded as basic approaches in this respect. It should further be noted that although the steps illustrated in FIGS. 2 and 3 are presented in a particular order, the steps need not necessarily be carried out in this order. For example, referring to FIG. 2 the region of interest detector may first determine a deviation for each sub-volume within the volume data before checking whether the respective deviations thus obtained are within the margin of deviation, or not.

Although a drawing shows different functional entities as different blocks, this by no means excludes implementations in which a single entity carries out several functions, or in which several entities carry out a single function. In this respect, the drawings are very diagrammatic. For example, referring to FIG. 1, the region of interest detector RDT and the slice generator SLG may be implemented by means of a single processor, which also implements the controller CTRL.

There are numerous ways of implementing functional entities by means of hardware or software, or a combination of both. As mentioned hereinbefore with reference to FIG. 1, the ultrasound scanning assembly USC, the region of interest detector RDT and the slice generator SLG are functional entities that may each be implemented by means of a set of instructions that has been loaded into a programmable processor. In this respect, FIG. 1 can be regarded to represent a method, whereby the ultrasound scanning assembly USC represents an ultrasound scanning step, the region of interest detector RDT represents a region of interest detection step, and the slice generator SLG represents a slice generation step. Although software-based implementations of these functional entities have been mentioned, hardware-based implementations are by no means excluded. Hardware-based implementations typically involve dedicated circuits, each of which has a particular topology that defines operations, which the dedicated circuit concerned carries out. Hybrid implementations are also possible in the sense that a system, or a functional entity comprises therein, comprises one or more dedicated circuits as well as one or more suitably programmed processors.

There are numerous ways of storing and distributing a set of instructions, that is, software, which allows an ultrasound imaging system to operate in accordance with the invention. For example, software may be stored in a suitable medium, such as an optical disk or a memory circuit. A medium in which software stored may be supplied as an individual product or together with another product, which may execute software. Such a medium may also be part of a product that enables software to be executed. Software may also be distributed via communication networks, which may be wired, wireless, or hybrid. For example, software may be distributed via the Internet. Software may be made available for download by means of a server. Downloading may be subject to a payment.

The remarks made herein before demonstrate that the detailed description with reference to the drawings, illustrate rather than limit the invention. There are numerous alternatives, which fall within the scope of the appended claims. Any reference sign in a claim should not be construed as limiting the claim. The word “comprising” does not exclude the presence of other elements or steps than those listed in a claim. The word “a” or “an” preceding an element or step does not exclude the presence of a plurality of such elements or steps. The mere fact that respective dependent claims define respective additional features, does not exclude a combination of additional features, which corresponds to a combination of dependent claims.

1. An ultrasound imaging system comprising:
an ultrasound scanning assembly arranged to provide volume data resulting from a three-dimensional scan of a body;
a region of interest detector arranged to detect a region within the volume data characterized by a variation of at least one data parameter, which exceeds a margin, by comparison of a set of local values, which have been determined for the at least one parameter based on a portion of the volume data, with at least one of the following:
(a) a set of global values, which have been determined for the at least one parameter based on the volume data in its entirety; and
(b) another set of local values, which have been determined for the at least one parameter based on another, adjacent portion of the volume data.

2. An ultrasound imaging system according to claim 1, comprising:
a slice generator arranged to generate slices from the region that has been detected, which slices can be displayed on a display device.

3. An ultrasound imaging system according to claim 1, comprising an interface via which an operator can specify the at least one data parameter used for detecting a region of interest.

4. An ultrasound imaging system according to claim 1, wherein the at least one data parameter used for detecting a region of interest comprises a data parameter selected from the following group: average voxel magnitude, contrast, entropy, homogeneity.

5. An ultrasound imaging system according to claim 1, the at least one data parameter comprising a set of parameters in the form of a histogram.

6-7. (canceled)

8. A method of ultrasound imaging involving an ultrasound scanning assembly arranged to provide volume data resulting from a three-dimensional scan of a body, the method comprising:
a region of interest detection step in which a region of interest within the volume data characterized by a variation of at least one data parameter, which exceeds a margin, is detected by comparison of a set of local values, which have been determined for the at least one parameter based on a portion of the volume data, with at least one of the following:
(a) a set of global values, which have been determined for the at least one parameter based on the volume data in its entirety; and
(b) another set of local values, which have been determined for the at least one parameter based on another, adjacent portion of the volume data.

9. A method of ultrasound imaging as claimed in claim 8, comprising:
a slice generation step in which slices are generated from the region that has been detected, which slices can be displayed on a display device.

10. A computer program product for an ultrasound imaging system comprising
an ultrasound scanning assembly arranged to provide volume data resulting from a three-dimensional scan of a body:
a programmable processor,
the computer program product comprising a set of instructions, which when loaded into the programmable processor, enables the programmable processor to carry out a region of interest detection step in which a region of interest within the volume data characterized by a variation of at least one data parameter, which exceeds a margin, is detected by comparison of a set of local values, which have been determined for the at least one parameter based on a portion of the volume data, with at least one of the following:
a) a set of global values, which have been determined for the at least one parameter based on the volume data in its entirety; and
b) another set of local values, which have been determined for the at least one parameter based on another, adjacent portion of the volume data.
